(19) United States
(12) Patent Application Publication
(10) Pub. No.: US 2011/0112527 A1
(43) Pub. Date: May 12, 2011

(54) FLEXIBLE MEDICAL ABLATION DEVICE AND METHOD OF USE

(75) Inventors: William C. Hamilton, JR., Queensbury, NY (US); Giorgio di Palma, Queensbury, NY (US)

(73) Assignee: AngioDynamics, Inc., Queensbury, NY (US)

(21) Appl. No.: 12/613,929

(22) Filed: Nov. 6, 2009

(51) Int. Cl. A61B 18/14

(52) U.S. Cl. 606/41

(57) ABSTRACT

Disclosed herein are methods and devices involving a medical probe placeable into tissue where the probe has a high pushability yet is capable of being conformed to a patient's shape due to use of a removable stiffener and a flexible needle section allowing for placement, imaging, and treatment to be performed without removal of the probe regardless of environmental and physical restrictions related to devices used during the patient's procedure.

### BACKGROUND OF THE INVENTION

1. Field of the Invention

The present invention relates generally to methods and devices involving a medical device with a flexible needle section where the device is capable of providing tissue treatment. More specifically, the invention relates to devices and methods regarding a device designed to be capable of significant pushability for placement as well as significant flexibility allowing for the device profile to be altered so as to allow maximum compatibility for use with medical devices and procedures including imaging.

2. Description of the Related Art

Comprehensive medical care involves utilization of a significant number of simultaneously coordinated technologies that must be compatible to be effective. For example technology for tissue treatment (including tissue ablation) must be used in conjunction with advanced visualization systems. For example, imaging technologies or imaging devices can be used to determine the position of an energy delivery component (such as a probe) of a tissue treatment system or device for tissue treatment (for clarity, herein the terms “tissue treatment system,” and the term “device for tissue treatment,” and the term “probe” may be used interchangeably, additionally, the term “probe device” may be used interchangeably with the previous 3 terms). These visualization systems must be used effectively with the tissue treatment devices to effect repositioning as necessary, and to determine volumes of treated tissue to make conclusions regarding efficacy of treatment.

Current tissue treatment systems involve technologies such as radiofrequency ablation (RF), thermal electric heating, focused ultrasound, cryotherapies, laser treatment, microwave, and traditional heating methods (including heated fluids) with electrodes using direct current or alternating current. In addition, irreversible electroporation (IRE) is one of the more recent tools of tissue treatment.

Current visualization systems used for patient care include imaging systems such as computed tomography, X-ray, and magnetic resonance imaging systems which in certain cases involve patients being placed in tubes for scans for all or a portion of their bodies. Hereinafter, when referring to visualization or imaging, referring to placing a patient in (or within) a tube is used interchangeably with placement in or into a bore for imaging.

In many cases the tissue treatment system cannot be used effectively in conjunction with the imaging device; for example when a patient is of a large body mass index, multiple problems exist that are not solved by current technology; the distance between the patient and the imaging tube may be minimal so tissue treatment system energy delivery components cannot be placed into the patient and left there while the patient is in the imaging device because of spatial limitations. This is important because ideally imaging of the probes is performed after placement but prior to ablation of tissue to ensure exact, proper positioning to affect the targeted region with specificity. In addition, to be effectively placed within the patient skin, the energy delivery component of the tissue treatment system must be rigid in order allow penetration into the skin and through tissue such as membranes, connective tissue, or muscle. A system that is solely rigid will not be compatible with varying shapes of patients and sizes of visualization systems; at the same time a system that cannot hold shape will be unable to allow effective positioning of the energy delivery component within the patient.

It is a purpose of this invention to overcome external geometrical concerns for placement of the device having significant pushability and flexibility. The invention allows for adequate placement of an energy delivery component through a patient's skin and positioning of a patient within a tube of a visualization device without the need for removal of the energy delivery component. The device allows users to account for physical spatial limitations that up until now have made other devices and imaging machines currently incompatible.

The invention provides for a device that can be rigid and flexible as needed to provide stable and secure probe placement yet allowing significant external range of motion to ensure compatibility of use.

The invention provides for use with described current tissue treatment systems (radiofrequency ablation (RF), thermal electric heating, focused ultrasound, cryotherapies, laser treatment, microwave, and traditional heating methods (including heated fluids) with electrodes using direct current or alternating current).

The invention also provides for use with a more recent tool of tissue treatment, namely electroporation including irreversible electroporation (each of nonthermal and thermal). Irreversible electroporation (IRE) is an invaluable recent tool of medical science for the treatment of tissue. IRE is a novel method of tissue treatment that involves nonthermal application of an electric field to transiently permeabilize cells using a method known as irreversible electroporation. Irreversible electroporation is a novel method of applying electrical fields across tissue through a delivery of pulses that effectively result in membrane permeabilization and in cell necrosis. The invention provides for compatibility of visualization devices with IRE treatment in a way not currently available by currently designed IRE devices.

### BRIEF SUMMARY OF THE INVENTION

The present invention provides among other things the capability to bend and conform a section of a device for tissue treatment so that the probe can be placed at a designated location with the distal portion (including the tip) placed within a patient while more proximal portions external to the patient can conform to the patient's shape as necessary (can match the conformation of the patient), and both the patient and probe can be positioned within a machine capable of visualizing at least a portion of the patient as well as the probe without the need for removal of the probe. These capabilities allow for use of the probe with additional medical devices. In one exemplary embodiment, the flexible section can be configured to be capable of bending so as to match the conformation (surface profile) of said patient from a point starting at the distal end of said handle of said probe to a point at the proximal end of said rigid needle section.

This invention allows the device to be used in situations where there is a physical limitation between the patient and a portion of a machine used for imaging such that the probe must be manipulated so as to change positions, if necessary lying even with the patients skin if the distance between a portion of the machine and the patient approaches a zero point. From here forth, the terms imaging machine and visualization machine will be used interchangeably throughout.

The invention provides for manipulation and movement so as to make the probe compatible for use with visualization devices known in the art such as computed tomography machines (CT), magnetic resonance imaging (MRI), X-Rays, or other imaging machines as well as techniques known in the art.

This invention provides for the physical manipulation of the flexible portion of the device so that it can allow adjustments for minor, major, or extreme angles and geometrical restrictions. It also allows for manipulation to decrease the profile of the probe.

This invention, in certain cases, allows for flexibility needle section to approximately attach to the tip itself as the rigid needle section length approaches zero.

This invention provides for having a stiffener placeable within the flexible portion of the probe in certain cases so as to allow for a rigidity at the time of placement within any organ limited only by the pushability of the stiffener, and to combine this with the maximum flexibility of the flexible portion which can rest on or near the patient's skin as necessary upon removal of the stiffener.

It is another object of this invention to have in certain embodiments a probe with a trocar tip such that the tip and the rigid portion are one piece, neither having an internal opening, bore, or working channel such that the pieces are as strong and stable as possible for placement of the probe in the patient for patient safety and treatment efficacy. This ensures the components do not break, separate, or conform improperly during placement and use, and ensures safe, determinable current application to tissue.

In certain embodiments the flexibility of the probe is utilized to overcome physical external restrictions of the visualization machine tube. In other words when there is not enough distance between the individual patient placed within the bore of the machine and the tube of the visualization machine, the flexibility of the probe allows scanning with the probe in place within the patient.

This invention provides for a reliable, effective and easy method to position a probe within a patient, to utilize an imaging machine to view the probe or patient or determine the position of the probe, and to apply pulses that can result in cell alteration through treatment or ablation all without having to remove the probe from the patient and while being able to manipulate the portions of the probe outside the patient to necessary positions to overcome external restrictions. In certain embodiments the release of pulses can be referred to as pulsed electric field gradients to the selected tissue.

This invention allows a physician to place a probe into the ideal and correct point of the skin or on or within other treatment regions at the ideal angle regardless of the external equipment for procedural imaging machines or additional equipment necessary for patient health or procedure success. In various embodiments the IRE application is nonthermal. In various embodiments the pulses are delivered so as to ensure that the temperature of the tissue does not exceed 50° C.

This invention provides for imaging and probe utilization without the need for removal of the probe from the patient in applications related to percutaneous, laparoscopic, open surgical, or procedures relating to natural orifices.

The invention provides for these and other purposes, objects, and applications using devices and methods involving a design wherein there is an electrical coupling coupled to a housing, in certain cases a strain relief, a flexible needle section, a rigid needle section, and a tip that is can be capable of piercing tissue, and wherein pushability can be supplied with a stiffener. For clarity, hereforth the term “housing” and the term “handle” may be used interchangeably. In one aspect, the elongated body can comprise the flexible needle section and the rigid needle section.

More specifically, the invention provides for these and other purposes, objects, and applications in part through a design wherein a flexible needle section is comprised of a coil or a cut metal coupled to the electrical coupling such as a wire from a generator and coupled to the tip, in certain cases through a rigid needle section, such that energy is capable of flowing through the flexible needle section, wherein the probe is configurable so as to conform with the patient's skin surface as necessary to avoid external machines, devices, or mechanisms. The stiffener provides for pushability so that the probe can have stability and be effective for placement in substantially any organ system and through the skin, and since the stiffener is removable, the probe has maximum stability and maximum flexibility. For clarity and as previously indicated, embodiments of the invention have a flexible portion made of cut metal, and a cut metal is a metal where material has been removed from parts of the metal so as to make the metal flexible; in certain embodiments a single continuous cut is made to remove material, and in other embodiments there are a series of cuts. The removals or cuts can be equally spaced in some embodiments and can be unequally spaced in other embodiments. The removal can be via mechanical or chemical methods. A laser can be used to remove material, as in a laser-cut.

The invention also provides for these and other purposes, objects, and applications in part through a design wherein a rigid needle section without any working channels or orifices or openings can be made as a single, unified component in certain cases with a tissue piercing tip and wherein the rigid needle section can be in direct contact with a stiffener to provide maximum pushability and still allow necessary flexibility upon removal of the stiffener.

The invention provides for these and other purposes, objects, and applications in part through a design having clear methods of use. The flexible probe can be utilized in certain embodiments as follows: the probe is coupled to the generator for treatment such as IRE. The probe is inserted into the patient. If necessary, the stiffener is removed from the probe and the flexible portion can be moved or even placed directly against the patient as necessary. The person is placed within the visualization or imaging machine. The flexible probe can be attached to the patient or other materials to ensure the probe is securely in place though this is not always required. An image is taken of the patient to ensure proper positioning of the probe. Optionally, treatment can be performed using real-time imaging with the probe in place within the targeted tissue region. If needed, the probe can be repositioned prior to a first treatment or after, and can be repositioned for a second or additional treatment. The stiffener can be replaced and removed as needed, with treatment and retreatment being performed as required. Reimaging can also be performed. The patient is removed from the imaging or visualization machine, the energy is turned off, and the probe is removed from the patient. Imaging machines are examples of the types of medical devices the probe is compatible with, though other medical devices and procedures requiring compatibility with probe placement and use are conceivable.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. §112, ¶ 6. Thus, the use of the words “function,” “means” or “step” in the Detailed Description Or Description of the Drawings or claims is not intended to somehow indicate a desire to invoke the special provisions of 35 U.S.C. §112, ¶ 6, to define the invention. To the contrary, if the provisions of 35 U.S.C. §112, ¶ 6 are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claim recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. §112, ¶ 6. Moreover, even if the provisions of 35 U.S.C. §112, ¶ 6 are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE SEVERAL VIEWS OF THE DRAWINGS

A more complete understanding of the present invention can be derived by referring to the detailed description when considered with the following illustrative figures. In the figures, like reference numbers refer to like elements or acts throughout the figures. Throughout the specification, the term “distal” is consistently used in reference to the device or portion of the device farthest from the user and “proximal” refers to the end closest to the user of the device.

FIGS. 1A and 1B are isometric views of a device designed for tissue treatment having a flexible needle section, a rigid needle section, and a tissue piercing tip. FIG. 1A shows the device with a stiffener in place providing structure while FIG. 1B shows a depiction where the stiffener has been removed.

FIG. 2 shows enlarged plan views of Detail 1A and Detail 1B of the device depicted in FIG. 1 showing an enlarged view of the distal end of the device for tissue treatment, specifically including the rigid needle section and the tissue piercing tip. Also shown in FIG. 2 is the interface between the flexible needle section and the rigid needle section.

FIG. 3 is a cross sectional view of the device depicted in FIG. 1 showing a stiffener in the interior of the probe.

FIG. 4 is an enlarged view of the cross section of FIG. 3, showing Detail 2A, Detail 2B, and Detail 2C showing the coil and wiring of the probe.

FIGS. 5A and 5B show various embodiments of a portion of the device depicted in FIG. 1 showing variations of the flexible needle section, including a coil in FIG. 5A and a portion of the flexible needle section comprised of cut metal in FIG. 5B.

FIG. 6 is a plan view of the device depicted in FIG. 1 showing the versatility and flexibility of the flexible needle section of the probe. In FIG. 6 the stiffener has been partly withdrawn from the probe.

FIG. 7 is a perspective view of the device depicted in FIG. 1 inserted into a region to be treated within a liver.

FIG. 8 shows a perspective view of the device depicted in FIG. 1 with the stiffener removed from the probe demonstrating the extreme angles possible with the flexible needle section allowing entry despite tight confines within a given medical environment.

FIGS. 9A, 98, and 9C show plan views of various embodiments of the device depicted in FIG. 1 demonstrating alternative designs of the voltage delivery region or regions of the probe. Shown are monopolar, bipolar, and array configurations.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention can be practiced without these specific details. In other instances, known structures and devices are shown or discussed more generally in order to avoid obscuring the invention. In many cases, a description of the operation is sufficient to enable one to implement the various forms of the invention. It should be noted that there are many different and alternative configurations, devices and technologies to which the disclosed inventions can be applied. The full scope of the inventions is not limited to the examples that are described below.

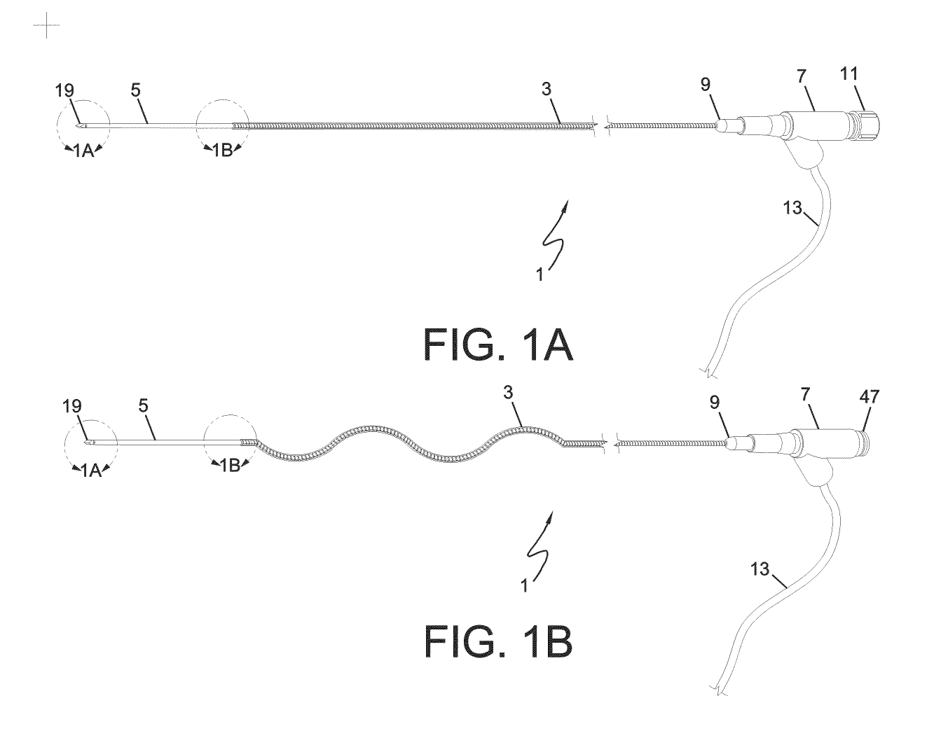

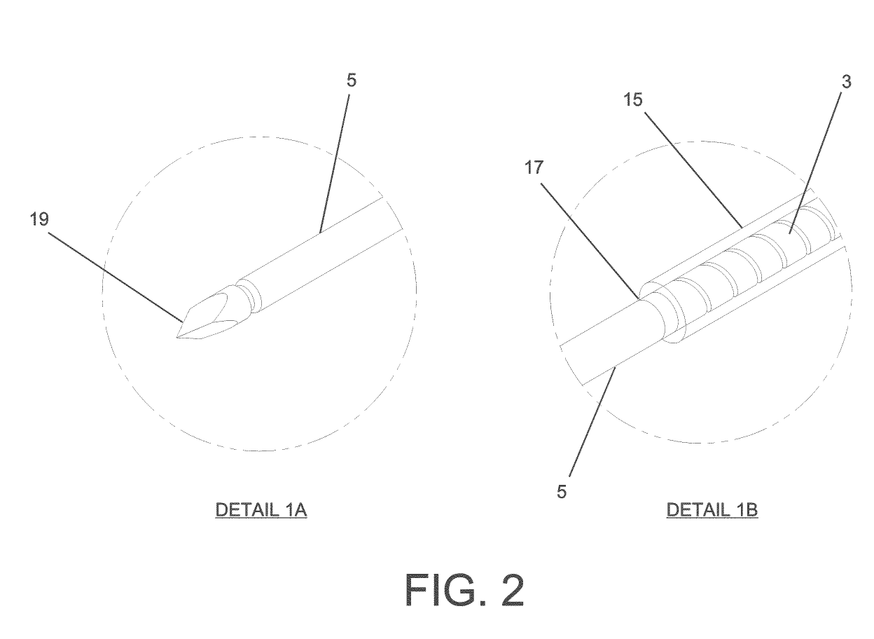

FIGS. 1A and 1B are plan views of a device designed for tissue treatment having a flexible needle section 3, a rigid needle section 5, and a tissue piercing tip 19. Shown is a device for tissue treatment 1. In certain cases the device for tissue treatment 1 can be called a probe device wherein the probe device comprises having an elongate body having a proximal end, a distal end, and a longitudinal axis extending between the proximal end and the distal end, wherein the body comprises a flexible section, a rigid section, and a tip, each having a proximal end and a distal end. The particular embodiment of the device for tissue treatment 1 shown in FIGS. 1A and 1B includes a longitudinal axis along which lies a flexible needle section 3, a rigid needle section 5, a housing 7 with a strain relief 9, and an electrical coupling 13. FIG. 1A shows a cap 11 which is located on the proximal end of a stiffener showing that in FIG. 1A there is rigidity throughout the device due to placement of the stiffener providing form and pushability throughout the flexible needle section 3. A stiffener is an object that can be placed into the lumen of the probe to provide form; as defined herein this can include but is not limited to a stylet, a rod, a wire, as well as a solid or hollow metal or plastic piece, each or any of which is either straight or not straight, with or without lumens centrally and with or without apertures on the side through which other wires or devices can move through in certain embodiments. In certain embodiments the flexible needle section has an inner lumen that is configured for the selective receipt of a stiffener. In one aspect, the lumen can be dimensioned for frictionally engaging an exterior surface of the stiffener. In another variation, the probe lumen can be dimensioned such that an annular space is created between the stiffener outer surface and the inner wall of the probe shaft. In one aspect, when the stiffener is inserted into the lumen of the probe, the stiffener and the lumen are positioned such that they are defined in a substantially coaxial relationship. In one aspect, when the stiffener is inserted into the lumen of the probe, the stiffener and the lumen are positioned such that they are defined in a substantially coaxial relationship. In FIG. 1B the stiffener has been removed from the probe; FIG. 18 shows a receiver for the cap 47 of the stiffener indicating the absence of a stiffener. The receiver for the cap 47 of the stiffener can be part of a luer connection or coupling. In certain embodiments the stiffener has a lumen or series of lumens, and in other embodiments has a lumen capable of receiving a guidewire. The rigid needle section, in certain embodiments, is up to 12 cm in length. In other embodiments, the rigid needle section is up to 4 cm in length, and in other embodiments, it is between 2 cm and 4 cm in length. In certain cases there is no strain relief. In other cases the strain relief is coupled to the flexible needle section using any attachment methods known in the art including but not limited to gluing. Detail 1A and Detail 1B are indicated in FIGS. 1A and 1B and are shown in expanded forms in FIG. 2 to more clearly depict the flexible needle section 3, the rigid needle section 5, and the tissue piercing tip 19.

FIG. 2 shows enlarged isometric partial view of Detail 1A and Detail 1B of the device in FIG. 1 showing an enlarged view of the distal end of the device for tissue treatment, specifically including the rigid needle section 5 and the tissue piercing tip 19 (Detail 1A). Also shown in FIG. 2 is the interface between the flexible needle section and the rigid needle section 17 (Detail 1B). More specifically, Detail 1A shows the rigid needle section 5 and the tissue piercing tip 19. The tissue piercing tip 19 can be of any type in the art necessary to place a probe for treatment, and can be sharp as well as hard to penetrate dense tissue or tissue difficult to penetrate; the tip can also be dulled or blunt to protect tissue as needed. The tip, in certain embodiments, has three sides or faces and in certain embodiments is machined so as to taper from the proximal to distal tip section, such that the most distal point of the tip is the thinnest section.

Detail 1B of FIG. 2 shows the interface 17 between the flexible needle section 3 and the rigid needle section 5. For completeness the insulation 15 surrounding the flexible needle section is also shown in Detail 1B. The insulation can be slideable and can be moved in certain embodiments using a controller that is part of the handle. The insulation sleeve, in certain embodiments, can be an insulation sleeve coaxially surrounding at least a portion of the flexible section. In certain embodiments the insulation is slideable along the rigid needle section so the exposed length of at least one active electrode can be set at between 0.01 centimeters and 4 centimeters. The controller for sliding the insulation can be mechanical or electrical in nature of any type known in the art. The insulation can be moved so that the rigid needle section is completely covered or completely bare regarding insulation. In certain embodiments, such as in certain monopolar embodiments, the active electrode is represented by the length of the tip only; in other embodiments the tip as well as the entire length of the rigid needle section acts as an active electrode. Therefore the active electrode in certain embodiments is up to 12 cm long, and in others is 4 cm, and in certain embodiments is 2 cm in length. Equivalent designs are conceivable for bipolar embodiments.

In certain embodiments the flexible needle section 3 and the rigid needle section 5 are coupled utilizing one or more than one of welding, soldering, or use of electrically conductive adhesive. Embodiments utilizing the cut metal can be made of a single piece where the rigid section remains intact without cuts and the flexible portion has had cuts or removed metal portions allowing for movement. This example embodiment of a single piece provides the advantage of stability and more certainty that the single piece will remain intact during and throughout use.

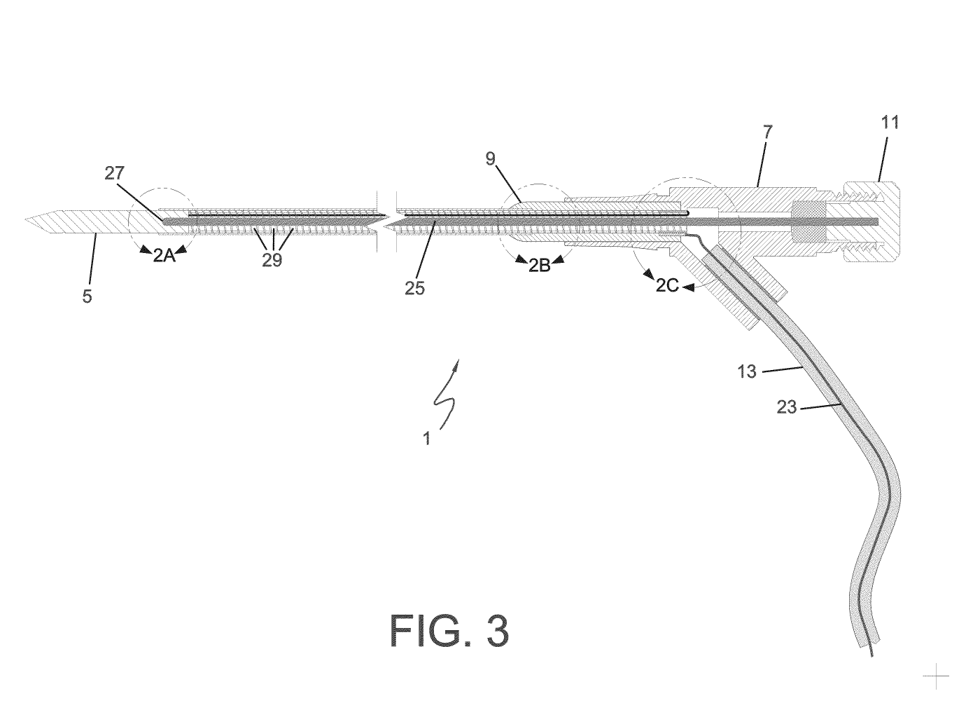

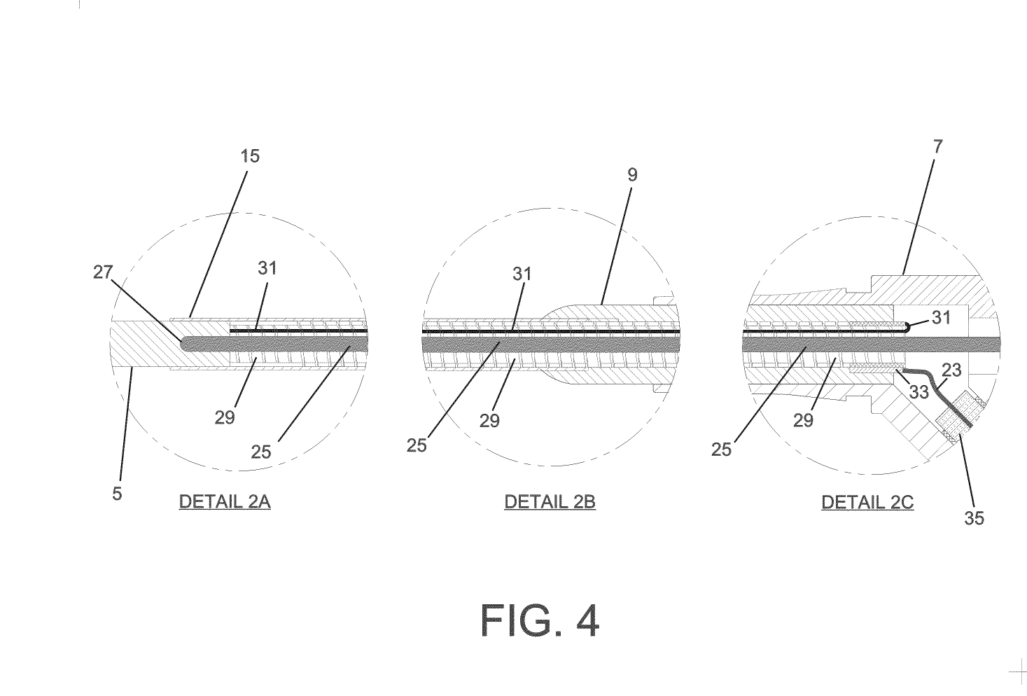

FIG. 3 is a cross sectional view of the probe of FIG. 1 showing a stiffener positioned within the lumen of the probe. Shown are the rigid needle section 5, the coil 29 and stiffener 25 (including the distal tip of the stiffener 27) as inserted through the device for tissue treatment 1 through the flexible needle section. Also indicated is the housing 7, the strain relief 9, the cap 11 of the stiffener, the electrical coupling 13 and the wiring of the electrical coupling 23 of the device for tissue treatment 1 which allows coupling to an energy source (not shown). Detail 2A and 2B and 2C are each indicated in FIG. 3 and shown in expanded forms in FIG. 4 to more clearly depict the internal components of a particular embodiment of the device for tissue treatment. The stiffener can be inserted through the flexible needle portion 3 until it comes in contact with the proximal portion of the rigid needle section 5. The distal end of the stiffener 27 and the proximal portion of the rigid needle section may optionally be designed to lock together or screw together so as to provide stability and rigidity to the device. This provides a distinct advantage where the device acts a single continuous piece from the tissue piercing lip to the cap at the proximal end of the stiffener. This allows better control and ease of positioning for the user.

The device can be monopolar in certain embodiments and the probe can be bipolar in certain embodiments. Monopolar involves a circuit with either an anode or cathode on a single probe; in that case use for a patient involves placement and activation of at least two monopolar probes or one probe and one grounding pad. Bipolar involves a circuit where there is at least one anode and at least one cathode on a single probe. In certain embodiments there could be more than two anodes or cathodes on a single probe. Monopolar and bipolar probes can be used individually or in combination to effectively treat or ablate tissue.

In various embodiments, energy can move from the generator through the electrical coupling 13, through the coil 29, and directly through the rigid needle section 5 including the tissue piercing tip 19. In certain embodiments the tip is a machined portion of the rigid needle section. In other cases the rigid needle section is solid having a continuous metal interior throughout the entire diameter and length of the rigid needle section. In various embodiments the rigid needle section contains a lumen or series of lumens, and in other embodiments the rigid needle section contains a lumen capable of receiving a guidewire. In other embodiments the tissue piercing tip contains a lumen, and in other embodiments the tissue piercing tip is capable of receiving a guidewire.

In certain embodiments the flexible region is comprised of a cut metal rather than a coil.

In example embodiments, the outer diameter of the probe is between 21 and 12 gauge. In certain embodiments the outer diameter is between 0.032 of an inch and 0.108 of an inch. One specific embodiment has an outer diameter of 0.072 of an inch.

The probe and the flexible portions of the probe can be of any length necessary for placement and use for treatment of a patient. In certain embodiments the flexible portion is from 2 to 12 centimeters in length. In other embodiments the flexible portion is up to 40 cm in length.

The coil is coupled to the rigid section by any method known in the art. Example methods of coupling include being glued or insert molded. The coil can be coupled to the wire from the generator by any method known in the art, including soldering or crimping, including coupling using a conductive plastic mechanically holding the parts in place. Controls for electroporation are in the generator and associated equipment to which the probe is coupled. In certain cases the electroporation may shut off on its own (such as depending on resistance levels that could indicate an unsafe condition). The coil can be shaped such that the loops of the wire are helical; in other embodiments the loops can be shaped as ovals, squares, triangles, or any other shape conceivable in the art still allowing for flexibility.

FIG. 4 is an enlarged view of the cross section of FIG. 3, showing Detail 2A, Detail 2B, and Detail 2C showing the coil and wiring of the probe. More specifically shown are the housing 7, strain relief 9, rigid needle section 5, coil 29, and insulation 15. Also shown is a stiffener 25 (with the distal tip of the stiffener 27 indicated), coil stabilization wire 31, wiring of the electrical coupling 23, wire placement coupling 33, and insulation of the electrical coupling 35. In certain embodiments the insulation 15 is made such that it is adjustable as to position or length or both. A mechanical or electrical mechanism on the handle or housing can be used to manipulate the position of the insulation. In certain embodiments there is a switch that when pushed in a distal direction on the handle, moves the insulation 15 in a distal direction, and when the switch is moved proximally, the insulation 15 moves proximally along the device.

In certain embodiments the flexible portion of the probe has insulation that is slideable using a switch, toggle, button, or other electrical or mechanical methods to move the insulation distally and proximally. The insulation in certain embodiments is an insulating plastic. In other embodiments the insulation is silicone, and in others, is Teflon. The insulation in certain embodiments is flexible. In example embodiments the thickness of the insulation is 0.003 inches, though any thickness necessary for safe use as known in the arts is conceivable. In other examples, the insulation is up to 0.01 inches thick. In others, the insulation is made of polyimide, and in yet other embodiments the insulation is made of polyamide. The insulation can be directly movable via a mechanical sliding.

The coil stabilization wire 31 extends from the housing 7 to the rigid needle section 5 and keeps the coil from unwinding. The coil stabilization wire keeps the coil from pulling apart so the coil does not open up. In specific embodiments the coil is made of stainless steel. In other embodiments the coil is made of a conductive plastic, including plastics with iron or silver additives. Conceivable embodiments include coils made of any conductive material known in the art, including those resistant to humidity as well as those resistant to rust. The coil stabilization wire can be substantially the same length as the coil. Part 33, the wire placement coupling, is shown in FIG. 4. In certain embodiments the wire placement coupling is solder. In other embodiments there is no wire placement coupling and the electrical coupling is coupled directly to the coil. The coil stabilization wire can be made of metal or conductive plastic or be made of a nonconductive material. In example embodiments the coil stabilization wire is composed of stainless steel or other metal or solder, or a combination of one or more of these materials. Any size of coil stabilization wire necessary to perform its function of stabilization regarding the coil is conceivable. In a specific example embodiment the coil stabilization wire is from 3 to 5 thousandths of an inch thick.

Part 33, the wire placement coupling, is shown positioned within the handle 7 in FIG. 4. The wire placement coupling 33 provides, in certain embodiments, a connection between the wiring of the electrical coupling 23 and the coil 29 for the transmission of energy from the generator to the exposed rigid needle section 5. Insulation 15 insulates the coil 29 and coil stabilization wire 31 during the application of energy.

The stiffener can be made of any material necessary to allow adequate pushability to perform necessary placement for probe utilization. The stiffener can be made or any material known in the art for stiffeners. In a specific example the stiffener is made of stainless steel. In one embodiment the stiffener may be a helically wound ribbon stiffener. In another specific example the stiffener has pushability equivalent to that of a 20 gauge biopsy needle. The pushability of the stiffener can be that necessary to place a portion of the probe within the tissue of interest, including placement into any treatable body portion; for example placement through skin, into an organ such as liver or lung, or through connective or bone tissue.

The stiffener can in certain cases be rigid and unbending. In other cases the stiffener has pushability and can bend to a limit less than that of the flexible portion of the probe. In certain cases after bending the stiffener will remain in the shape into which it has been bent. In other cases the stiffener, after bending, will rebound to its initial shape. The clearance between the coil and stiffener can be any distance necessary for proper use of the probe. In certain cases the distance between the coil and stiffener is between from about zero to 0.012 inches.

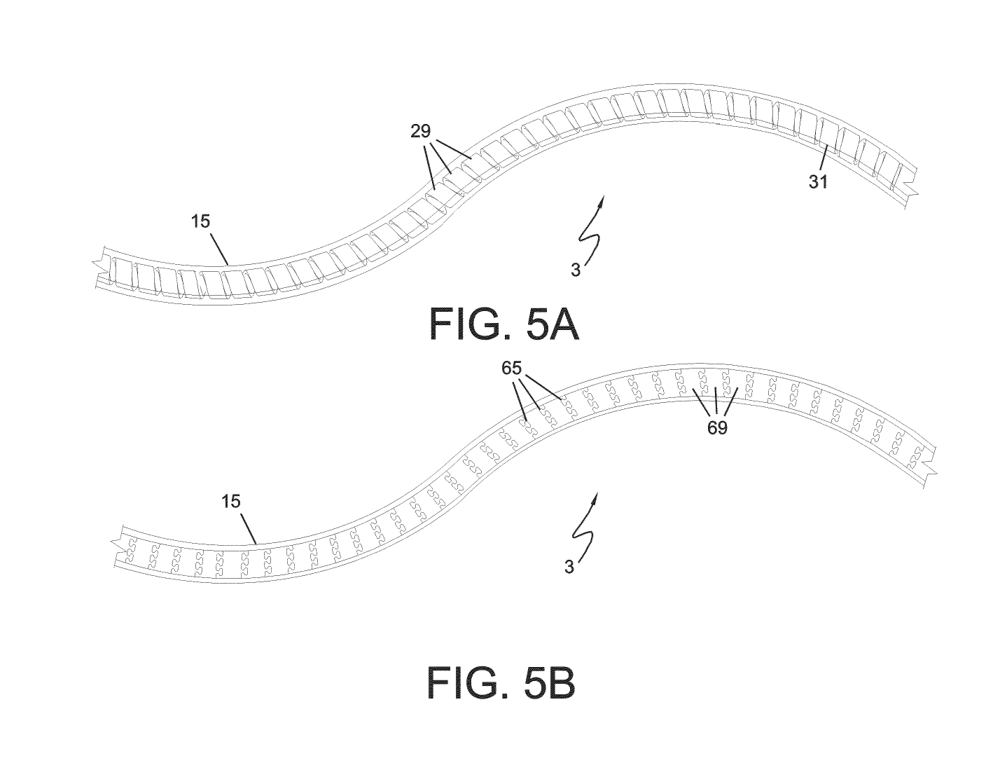

FIGS. 5A and 5B show various embodiments of a portion of the device depicted in FIG. 1 showing variations of the flexible needle section 3, including a coil in FIG. 5A and a section of the flexible needle section comprised of cut metal in FIG. 5B. Shown in FIG. 5A is a coil 29, insulation 15 surrounding the flexible needle section 3, and a coil stabilization wire 31. FIG. 5B shows the insulation 15 surrounding the flexible needle section 3, with metal sections 69 separated from each other by cuts in the metal 65. In other embodiments, the flexible section may be comprised of a flexible polymer material with a reinforced braiding embedded within the wall of the shaft. Other flexible shaft designs known in the art are also within the scope of the invention as long as the designs provide sufficient flexibility to conform to the patient when the stiffener is removed from the probe.

The diameter of the wire used for the coil is in certain cases from 0.03 to 0.012 of an inch. In a specific embodiment the wire is 0.007 of an inch in diameter. However the diameter of the wire can be any size necessary for proper probe functioning. In certain cases the wire is up to 0.036 of an inch in diameter.

In one example embodiment the inner diameter of the coil would be 0.058 of an inch where the outer diameter is 0.072 of an inch and the wire diameter is 0.007 of an inch; in that example the inner diameter has been calculated as 0.072 of an inch (the outer diameter) minus 0.014 of an inch (two times the diameter of the wire).

The coil can be made of round or flat wire. In certain embodiments there is substantially no space between each loop of the coil. In various embodiments the wire is kink-resistant or kink-proof.

In certain embodiments the flexible needle section is made of a conductive material, such as stainless steel, where a laser cut or chemical etching has been performed; in one example 2 thousandths of an inch of material thickness is removed for each cut, though the cut or cuts could be any diameter or depth as to allow maximum flexibility, including but not limited to from 2 to 100 thousandths of an inch of material removed with each cut. The cuts can involve a series of interlocking cuts. The distance between cuts can be of any distance necessary for flexibility necessary for probe placement and use. In certain embodiments the distance between the cuts are up to one quarter the length of the probe apart from each other. In other embodiments the cuts are below 1 cm apart, and in others they are below 0.1 cm apart.

The flexible needle section of the probe is bendable in any direction. In certain embodiments the range of bending is from 60-150 degrees. In other embodiments the coil could be bent more than 360 degrees.

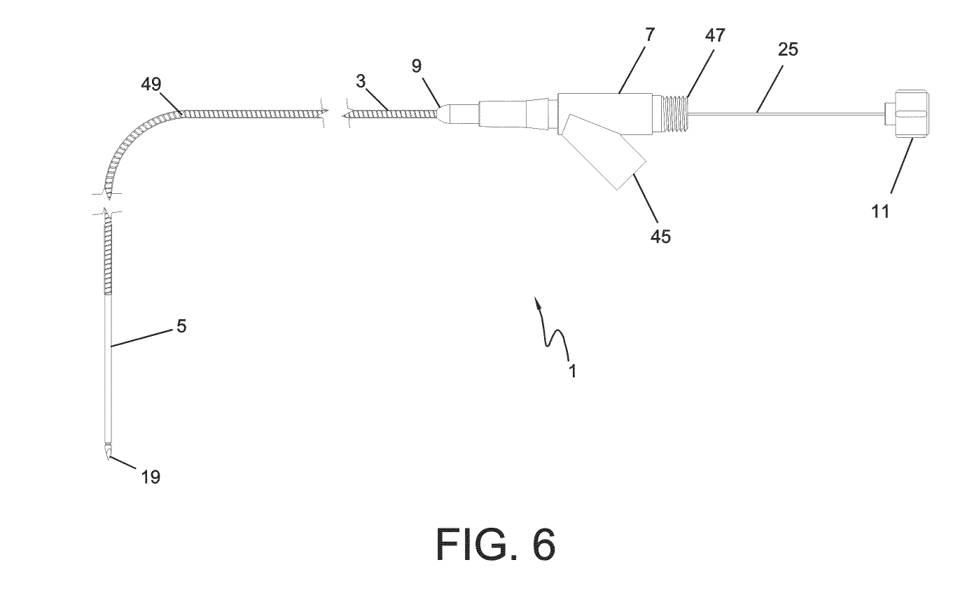

FIG. 6 is a plan view of the probe from FIG. 1 showing the versatility and flexibility of the flexible needle section of the device for tissue treatment 1. Shown is the flexible needle section 3, rigid needle section 5, tissue piercing tip 19, housing 7, strain relief 9, cap 11 of the stiffener, receiver for the cap 47, the stiffener 25, and the entry point 45 of the electrical coupling into the housing. In FIG. 6 the stiffener has been partially inserted through the flexible needle section 3 to demonstrate that the stiffener can provide stability and in certain embodiments rigidity to the device for that portion into which it is inserted; Point 49 shows the location along the flexible needle section 3 within which the most distal end of the stiffener would be located. Since the stiffener is only partially inserted, the flexible needle section 3 is shown in FIG. 6 as having rigidity from the point of the distal most portion of the strain relief 9 to point 49. The flexible needle section 3 is flexible for the portion more distal to the location of the distal end of the stiffener. Such flexibility is shown in FIG. 6 from point 49 to the most distal point of the flexible needs section 3 where it couples with the most proximal portion of the rigid needle section 5.

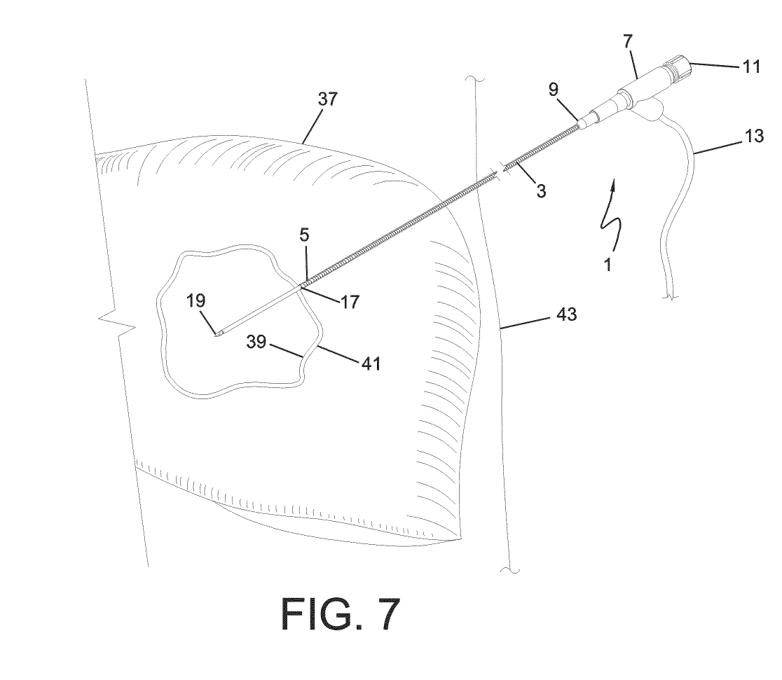

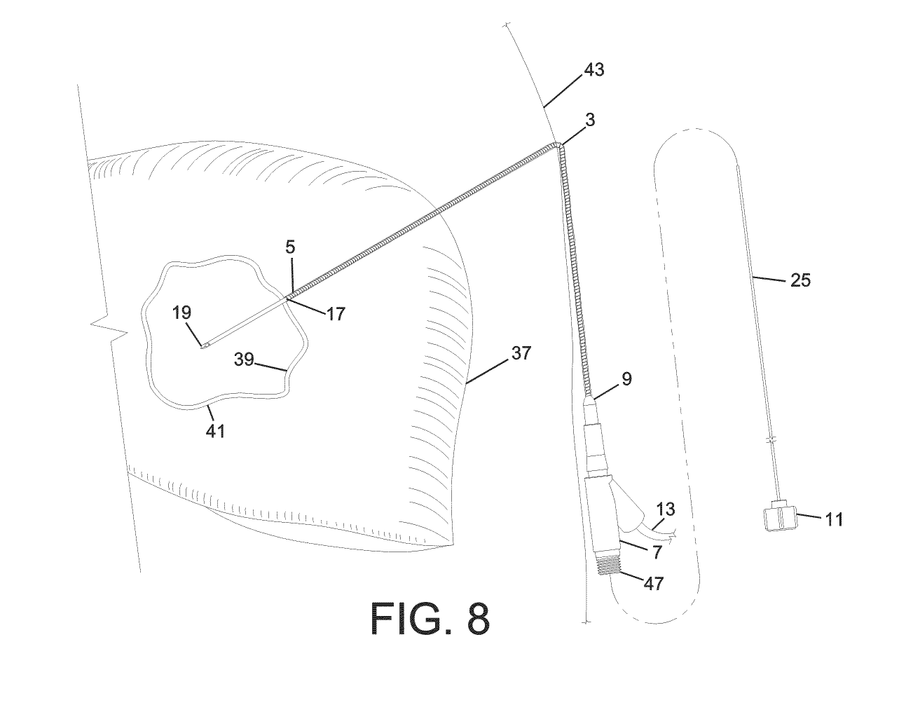

FIGS. 7 and 8 demonstrate examples of use of the device, and in certain embodiments the use can be described through the following method: 1) imaging of at least a portion of the patient as necessary to determine structure, boundaries of tissue to ablate, or status or tissue, 2) insertion of the stiffener into the probe to provide stability, 3) insertion of a portion of the probe into the patient, placing the probe into target tissue using the tissue piercing tip to advance the probe, 4) removing the stiffener and placing the flexible needle section in a shape to match the outline or profile of the patient, 5) ablating tissue, and 6) optionally, imaging again to determine results.

FIG. 7 is a perspective view of the probe from FIG. 1 inserted into a region to be treated within a liver. Shown is the device for tissue treatment 1, flexible needle section 3, rigid needle section 5, the interface 17 between the flexible needle section 3 and the rigid needle section 5, the housing 7, strain relief 9, cap 11 of the stiffener, electrical coupling 13, and a tissue piercing tip 19 of the device. Also shown is a liver 37 within a skin surface 43. The device for tissue treatment 1 is placed percutaneously through the skin and into the liver 37, with the tissue piercing tip 19 placed within a region to treat 39. Rigid needle section 5 in certain embodiments provides the active electrode section of the device during energy delivery. In cases of ablation, there is a safety margin surrounding the ablated region to assure complete ablation, so 41 indicates the region to treat as well as a safety zone surrounding the region to treat. In FIG. 7 the stiffener is shown inserted, including through the flexible needle section. The stiffener provides advantages such as providing necessary rigidity to insert the device percutaneously and advance to the desired location. The stiffener can also provide for enhanced visibility under imaging or when using imaging systems. The stiffener can provide added visibility during placement of the device.

FIG. 8 shows a perspective view of the probe from FIG. 1 demonstrating the extreme angles possible with the flexible needle section allowing entry despite tight confines within a given medical environment. The tissue piercing tip 19 of the device for tissue treatment is shown inserted into a region to be treated 39 within a liver 37. Also shown are the flexible needle section 3, rigid needle section 5, the interface 17 between the flexible needle section 3 and the rigid needle section 5, housing 7, strain relief 9, stiffener 25, cap 11 of the stiffener, and receiver for the cap 47. For perspective skin surface 43 is indicated. For clarity, the device for tissue treatment is placed within the liver 37, with the tissue piercing tip 19 placed within a region to treat 39. In cases of ablation, there is a safety margin surrounding the ablated region to assure complete ablation, so 41 indicates the region to treat as well as a safety zone surrounding the region to treat.

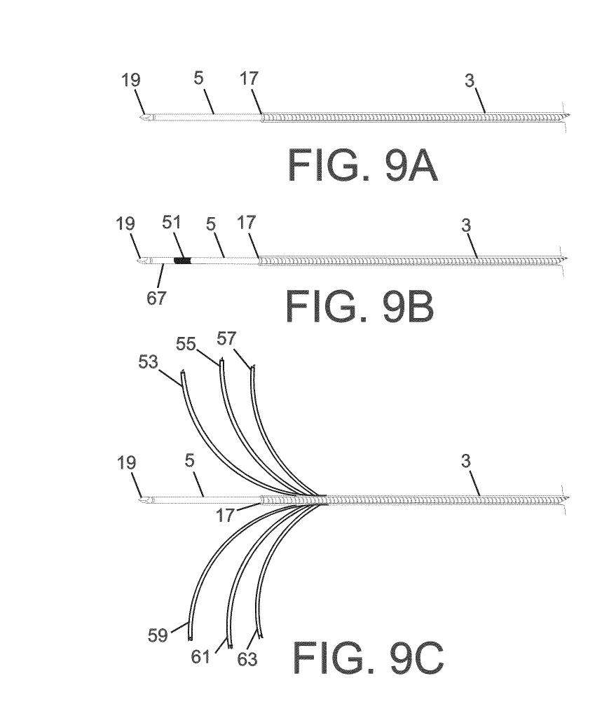

FIGS. 9A, 9B, and 9C show plan views of various embodiments of the device depicted in FIG. 1 demonstrating alternative variations of the organization of the voltage delivery region or regions of the probes. FIGS. 9A, 9B, and 9C each show embodiments of the device and show a flexible needle section 3, a rigid needle section 5, the interface 17 between the flexible needle section 3 and the rigid needle section 5, and a tissue piercing tip 19. FIG. 9B shows an electrically insulating region 67 that separates a voltage delivery region 51 from the tissue piercing tip 19 that can also act as a voltage delivery region; the electrically insulating region 67 acts in a manner sufficient (such as having a length sufficient) to prevent electrical shorting as well as to prevent arcing between voltage delivery regions. FIG. 9C shows an embodiment where multiple electrodes 53, 55, 57, 59, 61, 63 are capable of deployment and retraction through apertures in the flexible needle section 3. The embodiments in FIG. 9 illustrate that the device can be used to deliver energy via an electrode array in addition to monopolar and bipolar embodiments previously described.

FIG. 9A is an example of a monopolar embodiment of a device and can be utilized for tissue treatment using at least two monopolar embodiments or a monopolar probe with a grounding pad or other embodiment herein described. FIG. 98 shows a bipolar embodiment where an electrically insulating region 67 has separated two voltage delivery regions (51, 19). Additional voltage delivery regions on the rigid needle section 5 are conceivable. FIG. 9C shows an embodiment with electrodes deployed through the flexible needle section 3. An alternative embodiment can have the electrodes deployed through apertures on the side of the rigid needle section in embodiments where there is also a lumen in the rigid needle section running lengthwise. In certain embodiments the electrodes can be deployed physically such as by insertion of the stiffener. The electrodes can be deployed physically such as by insertion of the stiffener. Retraction could be via removal of the stiffener or via a string or other mechanism capable of being pulled, or a system could be used with another attachment between the electrodes and the stiffener. Deployment and retraction of the electrodes could be performed via a mechanical or electrical switch or other mechanism that is part of or attached to the handle. The electrodes can also be deployed and retracted using a hydraulic piston driving a fluid. Alternatively the stiffener can have shape such that the distal end of the stiffener locked or screwed into the proximal end of the antenna or antennas allowing the stiffener and antennas to be pushed and pulled together and yet be detachable from each other. Alternatively a torque coil can be placed inside the coil (29) and the torque coil can be turned to then affect the position of the electrodes. In addition, the coil can be replaced with a braided tube flexible in bending but not in compression.

The device and method of this invention can be used in laparoscopic, percutaneous, natural orifice procedures (NOTES), as well as open surgical procedures. The device and method of this invention can also be used when the target tissue either actually is one of the following tissues or is within the following tissues: digestive, skeletal, muscular, nervous, endocrine, circulatory, reproductive, integumentary, lymphatic, urinary, and soft tissue. The method can be used to target tissue of or within a vessel, a liver, or lung tissue. The method can also be used singly or in combination in tissues that are in the pancreas, prostate, uterus, and brain. The method can also be used to target singly or in combination tissues that are benign, malignant, cancerous, neoplastic, preneoplastic, or tumorous.

Treatment of tissue using this invention can be achieved with an IRE generator as the power source, utilizing a standard wall outlet of 110 volts (v) or 230 v with a manually adjustable power supply depending on voltage. In certain embodiments the generator has the capability of being activated and utilized within a voltage range of 100 v to 10,000 v and be capable of being adjusted at 100 v intervals. The applied pulses in various embodiments is between 20 and 100 microseconds in length, and capable of being adjusted at 10 microsecond intervals. The probes can be utilized with a generator that can be programmable and capable of operating between 2 and 50 amps, with test ranges involving an even lower maximum where appropriate. Various embodiments involve IRE treatment using 90 pulses. Various embodiments use a maximum field strength of between 20 V/cm and 8000 V/cm, and various embodiments utilize a maximum filed strength between 400 V/cm to 3000 V/cm between electrodes or between an electrode and a grounding pad or between various probes or probe components. Other embodiments utilize between 1500 V/cm and 2500 V/cm. Pulses can be are applied in groups or pulse-trains where a group of 1 to 15 pulses are applied in succession followed by a gap of 0.5 to 10 seconds. Pulses can be delivered using probes, needles, and electrodes each of varying lengths suitable for use in not only with percutaneous and laparoscopic procedures, but with open surgical procedures as well. Pulse lengths in various embodiments are from 5 milliseconds to 62 seconds. Other embodiments use pulse lengths up to 200 microseconds. In yet other embodiments the pulse length is between 70 microseconds and 100 microseconds.

Additionally, various treatment embodiments and scenarios can involve 8 pulses with a maximum field strength between electrodes (or between probes or probe components) of 250 V/cm to 500 V/cm. Probes in certain embodiments are used with generators capable of working within a voltage range of 100 kV-300 kV operating with nano-second pulses with a maximum field strength of 2,000V/an to, and in excess of, 20,000V/cm between electrodes. The probes of various embodiments are capable of efficient use between 2,000V/cm and 20,000V/cm.

Additionally, various treatment embodiments can involve current tissue treatment systems utilizing technologies such as radiofrequency ablation (RF), electroporation (reversible and irreversible, nonthermal or thermal), thermal electric heating, focused ultrasound, cryotherapies, laser treatment, microwave, and traditional heating methods (including heated fluids) with electrodes using direct current or alternating current.

What is claimed is:

1. A probe device, wherein the probe device comprises:
an elongate body having a proximal end, a distal end, and a longitudinal axis extending between the proximal end and the distal end, wherein the body comprises:
a flexible section, a rigid section, and a tip, each having a proximal end and a distal end,
wherein at least a portion of the proximal end of the tip extends to at least a distal portion of the rigid section,
wherein at least a portion of the proximal end of the rigid section extends to at least a portion of the distal end of the flexible section, and
wherein the flexible section comprises at least one lumen; and
a handle, wherein the handle has a proximal end, a distal end, and a longitudinal axis, and wherein at least a portion of the distal end of the handle is attached to at least a portion of the proximal end of the flexible section.

2. The device of claim 1, wherein the flexible section comprises at least one helical coil.

3. The device of claim 1, wherein the flexible section comprises at least one metal.

4. The device of claim 1, wherein the lumen of the flexible section is disposed along substantially the entire length of the flexible section; and
wherein the lumen comprises an inner wall and an outer wall; and
wherein the lumen is configured for the selective receipt of a stiffener.

5. The device of claim 1, further comprising a stiffener, wherein the stiffener has a proximal end and a distal end.

6. The device of claim 5, wherein the distal end of the stiffener is configured to be selectively coupled to at least a portion of the proximal portion of the rigid section via a mechanism selected from the group consisting of: a lock and a screw.

7. The device of claim 1, wherein the rigid section is substantially solid, and wherein the rigid section comprises at least one metal.

8. The device of claim 1, wherein the tissue piercing tip and the rigid section comprise at least one single, continuous metal.

9. The device of claim 1, wherein the elongate comprises at least one single, continuous metal.

10. The device of claim 1, wherein the device further comprises an insulation sleeve, and wherein the insulation sleeve coaxially surrounds at least a portion of the flexible section.

11. The device of claim 10, wherein the insulation sleeve is configured to be slideably moveable along at least a portion of a longitudinal axis of the rigid section.

12. The device of claim 1, wherein the device further comprises at least one electrical coupling, and wherein the electrical coupling is selectively coupled to at least a portion of the proximal portion of the handle.

13. The device of claim 12, wherein the electrical coupling is configured for carrying an electric current from a generator to the probe device for irreversible electroporation of a target tissue.

14. The device of claim 1, wherein the device is selected from a group consisting of: a monopolar electrode, a bipolar electrode, and an electrode array.

15. The device of claim 1, wherein the device comprises at least one active electrode.

16. The device of claim 1, wherein the device is configured for use with an imaging machine, wherein the imaging machine is selected from the group consisting of: a computed tomography machine, a magnetic resonance imaging machine, and an X-ray machine.

17. A method of using a probe device comprising:
providing a probe device, wherein the probe device comprises:
an elongate body having a proximal end, a distal end, and a longitudinal axis extending between the proximal end and the distal end, wherein the body comprises:
a flexible section, a rigid section, and a tip, each having a proximal end and a distal end,
wherein at least a portion of the proximal end of the tip extends to at least a distal portion of the rigid section,
wherein at least a portion of the proximal end of the rigid section extends to at least a portion of the distal end of the flexible section, and
wherein the flexible section comprises at least one lumen;
a stiffener having a proximal and a distal end,
wherein at least a portion of the stiffener is positioned within at least a portion of the at least one lumen of the flexible section; and
a handle, wherein the handle has a proximal end, a distal end, and a longitudinal axis,
wherein at least a portion of the distal end of the handle is attached to at least a portion of the proximal end of the flexible section;
inserting at least a portion of the probe device within a selected tissue in a patient body;
delivering energy to the selected tissue in a patient body to ablate the selected tissue;
removing the stiffener from the lumen of the flexible section.

18. The method of claim 17, further comprising the positioning the flexible section such that it conforms to at least one contour of a surface area of the patient body.

19. The method of claim 17, wherein delivering energy to the selected tissue comprises treating the selected tissue using nonthermal irreversible electroporation.

20. The method of claim 17, wherein after the step of providing the probe device, the method further comprises providing an imaging device and positioning at least a portion of the patient's body within an imaging machine, wherein at least a portion of the imaging machine defines a space where the patient's body is positioned along a longitudinal axis defined by the space.

21. The method of claim 20, wherein after the step of positioning the device within a selected tissue in a patient, the method further comprises positioning the handle of the device such that the longitudinal axis of the handle is positioned substantially parallel to the longitudinal axis of the patients body.
